(19) United States
(12) Patent Application Publication
(10) Pub. No.: US 2009/0117585 A1
(43) Pub. Date: May 7, 2009

(54) ANTI-FUNGAL SCREENING METHOD

(76) Inventor: Cornelis Antonius Maria Jacobus Johannes Van Den Hondel, Leiden (NL)

Correspondence Address: BROWDY AND NEIMARK, P.L.L.C.;624 NINTH STREET, NW, SUITE 300, WASHINGTON, DC, 20001-5303, US

(21) Appl. No.: 11/995,810

(22) PCT Filed: Jul. 14, 2006
(86) PCT No.: PCT/NL2006/050179

§ 371 Date: Aug. 11, 2008

(30) Foreign Application Priority Data
Jul. 15, 2005 (EP) 05 106532.4

(51) Int. Cl. G01N 33/53; C12N 9/90; C07H 21/04; C12N 15/64; C12N 5/10

(52) U.S. Cl. 435/7.1; 435/233; 536/23.2; 435/320.1; 435/348; 435/359

(57) ABSTRACT

The present invention relates to a method for identification of anti-fungal agents and their mode of actions. In particular, it relates to cell wall disturbing anti-fungal agents and to an assay to identify them. More particularly, it relates to a screening method for the identification of an anti-fungal compound, which method comprises (i) contacting a potential anti-fungal compound with a polypeptide which is involved in cell wall synthesis; and then (ii) identifying the effect which the potential anti-fungal compound has on the activity of the polypeptide, whereby reduced polypeptide activity is indicative for anti-fungal activity of the potential anti-fungal compound. It also relates to an overexpressing host cell and to a kit for performing the assay.

### FIELD OF THE INVENTION

The present invention relates to the identification of a cell wall related anti-fungal target and methods of identification of anti-fungal agents using the identified target. In particular, the target relates to the process of protein glycosylation in fungi.

### BACKGROUND OF THE INVENTION

The cell wall of fungi is an essential component of the fungal cell. By interfering with the synthesis or assembly of the fungal cell, the cell will lyse and die and therefore the cell wall is an ideal anti-fungal target. The fungal cell wall contains several classes of macromolecules, including β1,3-glucan, β1,6-glucan, chitin, cell wall galactomannoproteins and in some cases α1,3 or α1,3-α1,4-glucan. The proper synthesis, the transport and presence of these components in the cell wall and the crosslinking of the several components to each other to form a rigid cell wall are essential. Thus anti-fungals that interfere with the synthesis and transport of one of these components or anti-fungals that interfere with the crosslinking of those compounds are interesting as antifungal agents. Anti-fungals are grouped into five groups on the basis of their site of action: (1) azoles, which inhibit the synthesis of ergosterol (the main fungal sterol); (2) polyenes, which bind to fungal membrane sterol, resulting in the formation of aqueous pores through which essential cytoplasmic materials leak out; (3) allylamines, which block ergosterol biosynthesis, leading to accumulation of squalene (which is toxic to the cells); (4) flucytosine, which inhibits protein synthesis and (5) candins (inhibitors of the fungal cell wall), which function by inhibiting the synthesis of beta 1,3-glucan (the major structural polymer of the cell wall) (Balkis et al., 2002, Drugs 62 (7): 1025-1040). Only this latter class of candins are anti-fungal that specifically inhibit cell wall biosynthesis.

Although the class of candins are an interesting and potential valuable anti-fungal drug there is clearly a need for additional drugs, because laboratory experiments using S. cerevisiae have shown that mutants resistant to candins can spontaneously arise. Despite the recent entrance of glucan synthase inhibitors in clinical trials, knowledge of mechanisms of resistance against candins in patients is lacking. Furthermore, candins display a poor anti-fungal activity towards some fungi eg. C. neoformans and its activity towards non-Aspergillus molds have not been established today. Finally, tolerance against candins have been reported through activation of the PKC1 signalling cascade which offers the fungal cell a pathway to become resistant to candins. Therefore is it clear that there is a need for additional anti-fungals.

An anti-fungal agent that interferes with fungal cell wall biosynthesis and acts at the outside of the cell is highly preferable, because fungal cells possess several mechanisms to remove anti-fungal agents from the cell, e.g. by exporting them via plasma membrane localized transporters, which also decrease the efficiency by which a antifungal can act.

Currently, new anti-fungal screens are based on in vitro assays to screen anti-fungal compounds to affect biosynthesis of the cell wall. WO2004/048604 claims a method for the identification of compounds that affect GPI-anchor biosynthesis, CA2218446 claims a method for the identification of anti-fungal, which inhibits beta1,6-glucan In addition, method are disclosed in the article, to identify anti-fungals in vitro (e.g. Cercosporamide (Sussman et al., Eukaryotic Cell 3(4): 932-943). These in vitro screens are relatively difficult to perform, are likely to identify only anti-fungal compound that act inside the cell and therefore have to cross the membrane, and molecules that inhibit a reaction in vitro, may not have that effect in vivo, which indicates negative aspects of in vitro screening.

The use of reporter strains to identify cell wall related antifungal targets and to screen for anti-fungal compounds in vivo have been claimed in WO03020922 and WO2004/057033.

### SHORT DESCRIPTION OF THE FIGURES

FIG. 1: phylogenetic tree of UDP-galactopyranose mutases of Aspergillus fumigatus (Afu); Aspergillus niger (Ang); Caenorhabditis elegans (Nematode) (Cel); Aspergillus (Emerciella) nidulans (Eni); Gibberella zeae (Gze); Leishmania major (Protist) (Lma); Magneporthe grisae (Mgr); Neurospora crassa (Ncr); Trypanosoma cruzi (Protist) (tcr); Usilago maydis (Uma).

FIG. 2: Disruption phenotype of the A. niger UDP-galactopyranose mutase knock out strain.

(A) Schematic representation of the glfA wild-type locus, the plasmid pΔglfa (=pΔ8660) used for disruption and the deleted glfA locus (ΔglfA). The 0.7 kb KpnI fragment from the 5′ region of the glfA ORF is used as a probe. (B) Genomic DNA was digested with HindIII. In the wild-type strain ((N402), a 8.0 kb hybridizing fragment is expected, versus a fragment of 9.7 kb in the glfA deletion strain. The two strains (#97 and #67) with a proper pattern are indicated. (C). Deletion of glfA leads to an high osmolarity remediable temperature sensitive growth defect and an increased sensitivity towards 0.005% SDS and increased sensitivity to 75 μl/ml Congo Red (CR) at all three temperatures tested. 10-fold dilutions of spores, starting with 1×10⁴ spores as the highest concentration were spotted on Minimal Aspergillus Medium (MM) containing either SDS or CR as indicated. Plates were grown for 4 days at the indicated temperature. The concentration of sorbitol used to remediate the temperature sensitive growth defect is 1.2 M.

### DETAILED DESCRIPTION

The present invention relates to the identification of an anti-fungal target and toto the development of methods for screening anti-fungal compounds against the target. It also relates to a kit for carrying out the method and identify the mode of action of anti-fungal compounds.

In a first aspect, the invention relates to a screening method for the identification of an anti-fungal compound. The method comprises: (i) contacting a potential anti-fungal compound with a polypeptide which is involved in cell wall synthesis; and subsequently (ii) identifying the effect which the potential anti-fungal compound has on the activity of the polypeptide, whereby reduced polypeptide activity is indicative for anti-fungal activity of the potential anti-fungal compound.

The screening method of the invention is less complicated than other screening methods and has the advantage that it enables high through put screening and screening can be performed outside the living cell, e.g. in simple and readily available microtitre plates. In addition, the screening method can be highly specific towards the isolation of compounds that inhibit or reduce UDP-galactopyranose mutase activity.

### Screening Method and Polypeptide

Any potential anti-fungal compound may be contacted with a polypeptide, which is involved in cell wall synthesis. The potential anti-fungal compound may be any compound, which is suspected to prevent or inhibit the proliferation of fungal cells, such as yeast or filamentous fungus. In particular anti-fungal compounds, which are suspected to prevent or inhibit the proliferation of filamentous fungi are preferred. To isolate new potential anti-fungal compound using the screening method of the invention, microbial, fungal or natural extracts may be used. Alternatively, chemical libraries may be used.

The polypeptide or protein, which is involved in cell wall synthesis can be any polypeptide known to play a role in cell wall synthesis and/or cell wall remodelling. The polypeptide may be important for the conversion of cell wall precursors into cell wall components. According to a preferred embodiment of the invention, the polypeptide is an enzyme, which is involved in the formation of cell wall polysaccharides. The polypeptide used in this method is neither alpha 1,3-glucan synthase (AgsA) nor glutamine-fructose-6-phosphate (GfaA) as identified in WO 03/020922. Surprisingly, in a more preferred embodiment, the polypeptide is an enzyme involved in the formation of cell wall galactomannan synthesis. In an even more preferred embodiment, the polypeptide is involved in the formation of the sugar galactofuranose, a characteristic component in the cell wall of filamentous fungi. According to an even more preferred embodiment, the polypeptide is involved in the formation of galactofuranose (Galf). Even more preferably, the polypeptide is a UDP-galactopyranose mutase (EC 5.4.99.9) enzyme. This enzyme catalyses the interconversion of the 6-membered sugar ring of UDP-galactopyranose and the 5-membered sugar ring of UDP-galactofuranose.

Preferred Polypeptides to be Used in the Screening Method and being the Object of the Invention as Such

Even more preferably, the polypeptide used in the screening method has UDP-galactopyranose mutase activity and an amino acid sequence which has at least 48% identity with the amino acid sequence of SEQ ID NO:1. This polypeptide as such having UDP-galactopyranose mutase activity and having at least 48% identity with the amino acid sequence of SEQ ID NO:1 is a further aspect of the invention. Bacterial UDP-galactopyranose mutase have already been isolated and partially characterized (Scherman et al. (2003) Antimicrobial Agents and Chemotherapy 47:378-382). Bacterial UDP-galactopyranose mutase have less than 48% identity with SEQ ID NO:1.

The amino acid sequence of UDP-galactopyranose mutase from Aspergillus niger is given in SEQ ID NO:1. It was derived from the genomic sequence which is represented by nucleotides 1533-3432 of SEQ ID NO:3 which is flanked by promoter and terminator regions. The cDNA sequence encoding the amino acid sequence of SEQ ID NO:1 is given in SEQ ID NO:2.

The activity of this enzyme is preferably measured using the assay described by Scherman et al, (2003) Antimicrobial Agents and Chemotherapy 47:378-382.

According to an even more preferred embodiment, the polypeptide has at least 50%, 55%, 60%, 65%, 70%, 75%, 80% or 85%, even more preferably at least 90%, 92%, 95%, 97%, 98% or 99% identity with the amino acid sequence of SEQ ID NO:1.

According to one preferred embodiment, the polypeptide comprises a fragment, said fragment having at least 50% identity with fragment 1, fragment 1 consisting of amino acid number 41 till amino acid number 111 of SEQ ID NO:1. Fragment 1 of SEQ ID NO:1 is delimited by the following amino acids: ETPGG . . . NNIS. According to an even more preferred embodiment, the polypeptide comprises a fragment, said fragment having at least 55%, 60%, 65%, 70%, 75%, 80% or 85%, even more preferably at least 90%, 92%, 95%, 97%, 98% or 99% identity with the fragment 1 as defined above.

According to another preferred embodiment, the polypeptide comprises a fragment, said fragment having at least 50% identity with fragment 2, fragment 2 consisting of amino acid number 301 till amino acid number 335 of SEQ ID NO:1. Fragment 2 of SEQ ID NO:1 is delimited by the following amino acids: GIRGT . . . NYS. According to an even more preferred embodiment, the polypeptide comprises a fragment, said fragment having at least 55%, 60%, 65%, 70%, 75%, 80% or 85%, even more preferably at least 90%, 92%, 95%, 97%, 98% or 99% identity with the fragment 2 as defined above.

According to another preferred embodiment, the polypeptide comprises two fragments, one fragment having at least 50% identity with fragment 1 and the other having at least 50% identity with fragment 2 as defined above. According to an even more preferred embodiment, the identity with each fragment is of at least 55%, 60%, 65%, 70%, 75%, 80% or 85%, even more preferably at least 90%, 92%, 95%, 97%, 98% or 99%. Most preferably, the identity with each fragment is 100%.

In one preferred embodiment, the polypeptide of the invention and used in the screening method of the invention comprises an amino acid sequence which is 100% identical to the amino acid sequence of SEQ ID NO:1. Most preferably, the polypeptide having the amino acid sequence of SEQ ID NO:1 or a polypeptide being obtainable by expression of the cDNA present in the E. coli DH5 α deposited under accession number CBS 120060 is used in the screening method. Accordingly, the polypeptide having the amino acid sequence of SEQ ID NO:1 or a polypeptide being obtainable by expression of the cDNA present in the E. coli DH5 α deposited under accession number CBS 120060 as such is also the preferred polypeptide of the invention. Percentage of identity is calculated as the number of identical amino acid residues between aligned sequences divided by the length of the aligned sequences minus the length of all the gaps. Multiple sequence alignment was performed using DNAman 4.0 optimal alignment program using default settings.

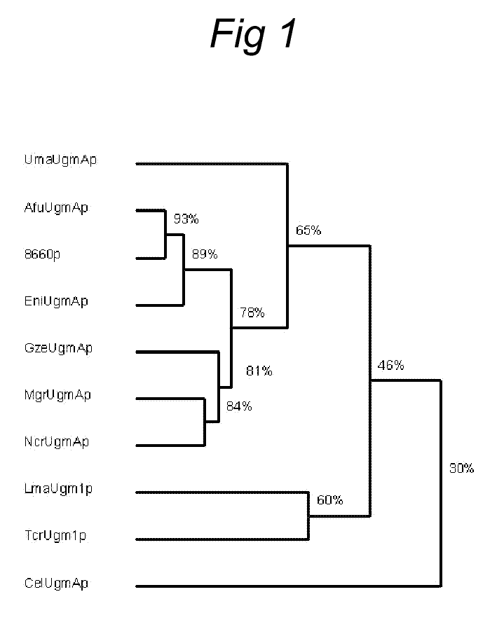

The skilled person will understand that the screening method of the invention could be applied using UDP-galactopyranose mutase enzymes obtained from other fungal organisms, eg, from other filamentous fungi. Preferred fungal organisms are Aspergillus fumigatus, Aspergillus flavus, Aspergillus parasiticus, Aspergillus nidulans, Aspergillus oryzae, Penicilium chrysogenum, Neurospora crassa, Trichoderma reesei, Trichoderma viridie, Chrysosporium lucknowense, Gibberella zeae (anamorph Fusarium graminarium), Cryptococcus neoformans, Coccidioides immitis, Magneporthe grisae Ustilago maydis. Any fungus expressing a UDP-galactopyranose mutase enzyme of the invention is a potential target of the potential anti-fungal compound tested in the method of the invention. Such polypeptides may be obtained using state of the art molecular biology techniques. Most preferably, the polypeptide used is obtained from an Aspergillus niger strain. It is also encompassed by the invention to isolate several UDP-galactopyranose mutase enzymes from one single organism. Accordingly, all these polypeptides are also as such part of the invention. FIG. 1 gives a phylogenetic tree for UDP-galactopyranose mutase from different eukaryotic organisms. The numbers indicate percentage of identity. Percentage of identity was determined by calculating the ratio of the number of identical amino acids in the sequence divided by the length of the amino acid sequence minus the lengths of any gaps. The numbers at the branches in the tree indicate the percentage identity. The protein multiple sequence alignment was performed using DNAman version 4.0 using the Optimal Alignment (Full Alignment) program.

According to another preferred embodiment, the polypeptide of the invention, which is also preferably used in the screening method of the invention is a variant of any one of the polypeptide sequences defined before. A variant polypeptide may be a non-naturally occurring form of the polypeptide. A polypeptide variant may differ in some engineered way from the polypeptide isolated from its native source. A variant may be made by site-directed mutagenesis starting from the amino acid sequence of SEQ ID NO:1 or from the nucleic acid sequence encoding the amino acid sequence of SEQ ID NO:1, which is SEQ ID NO:2. Preferably, the polypeptide variant contains mutations that do not alter the biological function of the encoded polypeptide. According to a preferred embodiment, the polypeptide variant has an enhanced UDP-galactopyranose mutase activity. A polypeptide variant with an enhanced UDP-galactopyranose mutase activity, is a polypeptide exhibiting an UDP-galactopyranose mutase activity, which is increased compared to the UDP-galactopyranose mutase activity of its wild type counterpart measured in a given assay. Preferably, the assay is the one described by Scherman et al, (2003) Antimicrobial Agents and Chemotherapy 47:378-382. According to a more preferred embodiment, the polypeptide variant has an enhanced UDP-galactopyranose mutase activity compared to the polypeptide having SEQ ID NO: 1 as measured in the Sherman assay as defined above. According to an even more preferred embodiment, the polypeptide variant has an enhanced UDP-galactopyranose mutase activity compared to the UDP-galactopyranose activity of Aspergillus niger ATCC9029 or CBS 120.49 and derivatives as preferably measured using the Scherman assay defined above. Polypeptides with enhanced activities are very useful since they can be advantageously used in the screening assay of the invention. It is expected that the screening assay would be more sensitive when this kind of variant polypeptide is being used.

According to a first preferred embodiment, contacting the polypeptide with a potential anti-fungal compound is carried out in vitro in a simple container, such as a microtitre plate. Alternatively, and according to a second preferred embodiment, contacting the polypeptide with a potential anti-fungal compound is carried out in vivo in a host cell as explained later on in the description.

In the second step of the method, the effect of the potential anti-fungal compound on the activity of the polypeptide is identified, whereby reduced polypeptide activity is indicative for anti-fungal activity of the potential anti-fungal compound. Polypeptide activity may be any detectable and measurable activities of a polypeptide, e.g. enzyme activity, inhibitory activity, biosynthetic activity, transporter activity, cell division activity, transcriptional activity or translational activity. According to a preferred embodiment, the activity of the polypeptide means enzymatic activity.

Depending on the identity of the polypeptide involved in cell wall synthesis used, the skilled person would know which assay is the best suited to assess the activity of the chosen polypeptide. The effect of the potential anti-fungal compound may be determined using an assay, preferably a microtitre plate assay, wherein the activity of the polypeptide is determined with any simple assay known to the skilled person, e.g. an assay based on radioactivity, fluorescence or by HPLC.

When polypeptide activity means enzymatic activity, enzymatic activity is preferably assessed using an assay detecting the conversion of UDP-galactopyranose into UDP-galactofuranose, especially if the polypeptide used in a UDP-galactopyranose mutase enzyme. A preferred assay to be used for the detection of UDP-galactopyranose mutase activity was described by Scherman et al, (2003) Antimicrobial Agents and Chemotherapy 47:378-382.

Other preferred assays include assays to screen for (conditional) mutants with reduced levels of Galf residues by antibody labelling using Galf specific antibodies, suicide selection methods using radioactive labelled Galf. These approaches would lead to the identification of other potential antifungal targets in relation the addition of GalF to O- or N-linked mannose chains. A preferred suicide selection methods using radioactive labelled Galf is carried out as has been described for the isolation of mannosylation mutants (Huffaker, T. C., and Robbins, P. W. (1982) J. Biol. Chem. 257, 3203-3210).

The reduction of polypeptide activity is assessed by testing the polypeptide activity in the presence and in the absence of the potential anti-fungal compound. Preferably, the activity of the polypeptide in the presence of the potential anti-fungal compound is reduced compared to the activity of the polypeptide in the absence of the potential anti-fungal compound. The polypeptide activity may be reduced completely, i.e. 100%, or in part. For instance, it may be reduced for more than 10%, 20%, 30%, 40%, 50%, 60% or 70%, or for more than 75%, 80%, 85% or 90% or for more than 92%, 94%, 96%, 98%, or 99%.

According to a preferred embodiment, a reduction of the activity of the polypeptide in the presence of the potential anti-fungal compound of at least 20% compared to the activity of the polypeptide in the absence of said potential anti-fungal compound is indicative for anti-fungal activity of the potential anti-fungal compound.

According to a more preferred embodiment, a reduction of the activity of the polypeptide in the presence of the potential anti-fungal compound of at least 30%, even more preferably of at least 40% and most preferably of at least 50% compared to the activity of the polypeptide in the absence of said potential anti-fungal compound is indicative for anti-fungal activity of the potential anti-fungal compound.

### Nucleic Acid Sequence

In a further aspect, the invention relates to a nucleic acid sequence coding for all the preferred polypeptides defined in the former section entitled “Preferred polypeptides to be used in the screening method and being object of the invention as such” as:
having UDP-galactopyranose mutase activity and
having an amino acid sequence which has at least 48% identity with the amino acid sequence of SEQ ID NO:1.

According to a preferred embodiment, the nucleic acid sequence is selected from the list consisting of:

(a) a nucleic acid sequence having at least 50% identity with the nucleic acid sequence of SEQ ID NO:2
(b) a variant of (a).

Percentage of identity was determined by calculating the ratio of the number of identical nucleotides in the sequence divided by the length of the total nucleotides minus the lengths of any gaps. DNA multiple sequence alignment was performed using DNAman version 4.0 using the Optimal Alignment (Full Alignment) program. The minimal length of a relevant DNA sequence showing 505% or higher identity level should be 40 nucleotides or longer. Preferably, the identity is of at least 55%, more preferably at least 60%, more preferably at least 65%, more preferably at least 70%, more preferably at least 75%, more preferably at least 80%, more preferably at least 85%, more preferably at least 90%, even more preferably at least 95%, even more preferably at least 98% and even more preferably at least 99%. Most preferably, the nucleic acid sequence has the nucleic acid sequence of SEQ ID NO:2. According to a preferred embodiment, the nucleic acid sequence comprises a fragment, said fragment having at least 50% identity with the fragment consisting of base pair number 121 till base pair number 334 of SEQ ID NO:2. This fragment of SEQ ID NO:2 is named fragment I. It codes for fragment 1 of the polypeptide as earlier defined. Preferably, the identity with fragment I is of at least 55%, more preferably at least 60%, more preferably at least 65%, more preferably at least 70%, more preferably at least 75%, more preferably at least 80%, more preferably at least 85%, more preferably at least 90%, even more preferably at least 95%, even more preferably at least 98% and even more preferably at least 99%. Most preferably, the identity with fragment I is 100%.

According to a preferred embodiment, the nucleic acid sequence comprises a fragment, said fragment having at least 50% identity with the fragment consisting of base pair number 901 till base pair number 1006 of SEQ ID NO:2. This fragment of SEQ ID NO:2 is named fragment II. It codes for fragment 2 of the polypeptide as earlier defined. Preferably, the identity with fragment II is of at least 55%, more preferably at least 60%, more preferably at least 65%, more preferably at least 70%, more preferably at least 75%, more preferably at least 80%, more preferably at least 85%, more preferably at least 90%, even more preferably at least 95%, even more preferably at least 98% and even more preferably at least 99%. Most preferably, the identity with fragment II is 100%.

According to a preferred embodiment, the nucleic acid sequence comprises two fragments, one fragment having at least 50% identity with fragment I and the other having at least 50% identity with fragment II as defined above. More preferably, the identity with each fragment is of at least 55%, more preferably at least 60%, more preferably at least 65%, more preferably at least 70%, more preferably at least 75%, more preferably at least 80%, more preferably at least 85%, more preferably at least 90%, even more preferably at least 95%, even more preferably at least 98% and even more preferably at least 99%. Most preferably, the identity with each fragment is 100%.

According to another preferred embodiment, the nucleic acid sequence of the invention is a variant of the nucleic acid sequence defined above. Nucleic acid sequence variants may be used for preparing polypeptide variants as defined earlier. A nucleic acid variant may be a fragment of any of the nucleic acid sequences as defined above. A nucleic acid variant may also be a nucleic acid sequence that differs from SEQ ID NO:2 by virtue of the degeneracy of the genetic code. A nucleic acid variant may also be an allelic variant of SEQ ID NO:2. An allelic variant denotes any of two or more alternative forms of a gene occupying the same chromosome locus. A preferred nucleic acid variant is a nucleic acid sequence, which contains_silent mutation(s). Alternatively or in combination, a nucleic acid variant may also be obtained by introduction of nucleotide substitutions, which do not give rise to another amino acid sequence of the polypeptide encoded by the nucleic acid sequence, but which corresponds to the codon usage of the host organism intended for production of the polypeptide of the invention. According to a preferred embodiment, the nucleic acid variant encodes a polypeptide still exhibiting its biological function. More preferably, the nucleic acid sequence variant encodes a polypeptide exhibiting UDP-galactopyranose mutase activity. Even more preferably, the nucleic acid variant encodes a polypeptide with enhanced UDP-galactopyranose mutase activity as defined earlier. Nucleic acid sequences encoding a polypeptide exhibiting UDP-galactopyranose mutase activity may be isolated from any microorganism.

All these variants can be obtained using techniques known to the skilled person, such as screening of library by hybridisation (southern blotting procedures) under low to medium to high hybridisation conditions with for the nucleic acid sequence SEQ ID NO:2 or a variant thereof which can be used to design a probe. Low to medium to high stringency conditions means prehybridization and hybridization at 42° C. in 5×SSPE, 0.3% SDS, 200 pg/ml sheared and denatured salmon sperm DNA, and either 25% 35% or 50% formamide for low to medium to high stringencies respectively. Subsequently, the hybridization reaction is washed three times for 30 minutes each using 2×SSC, 0.2% SDS and either 55° C., 65° C., or 75° C. for low to medium to high stringencies.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptide obtainable by expression of the cDNA present in the E. coli DH5 α deposited under accession number CBS 120060 containing the nucleic acid sequence coding for the polypeptide of the invention should prevail.

### Nucleic Acid Construct Expression Vector

In a further aspect, the invention relates to a nucleic acid construct comprising the nucleic acid sequence defined in the former section, said nucleic acid sequence encoding a polypeptide exhibiting UDP-galactopyranose mutase activity and having an amino acid sequence which has at least 65% identity with the amino acid sequence of SEQ ID NO:1. Optionally, the nucleic acid sequence present in the nucleic acid construct is operably linked to one or more control sequences, which direct the production of the polypeptide in a suitable expression host.

Operably linked is defined herein as a configuration in which a control sequence is appropriately placed at a position relative to the nucleic acid sequence coding for the polypeptide of the invention such that the control sequence directs the production of the polypeptide of the invention.

Expression will be understood to include any step involved in the production of the polypeptide including, but not limited to transcription, post-transcriptional modification, translation, post-translational modification and secretion.

Nucleic acid construct is defined as a nucleid acid molecule, which is isolated from a naturally occurring gene or which has been modified to contain segments of nucleic acid which are combined or juxtaposed in a manner which would not otherwise exist in nature. Control sequence is defined herein to include all components, which are necessary or advantageous for the expression of a polypeptide. At a minimum, the control sequences include a promoter and trancriptional and translational stop signals.

The invention also relates to expression vectors comprising the nucleic acid construct of the invention. Preferably, the expression vector comprises the nucleic acid sequence of the invention, which is operably linked to one or more control sequences, which direct the production of the encoded polypeptide in a suitable expression host. At a minimum control sequences include a promoter and transcriptional and translational stop signals. The expression vector may be seen as a recombinant expression vector. The expression vector may be any vector (e.g. plasmic, virus), which can be conveniently subjected to recombinant DNA procedures and can bring about the expression of the nucleic acid_sequence encoding the polypeptide. Depending on the identity of the host wherein this expression vector will be introduced and on the origin of the nucleic acid sequence of the invention, the skilled person will know how to choose the most suited expression vector and control sequences.

### Host Cell

In a further aspect, the present invention relates to a host cell, which comprises the nucleic acid construct or the expression vector of the invention as defined in the former paragraph. The host cell expresses the polypeptide of the invention having UDP-galactopyranose mutase activity and having an amino acid sequence which has at least 65% identity with the SEQ ID NO:1. The choice of the host cell will to a large extent depend upon the source of the nucleic acid sequence of the invention. Depending on the identity of the host cell, the skilled person would know how to transform it with the construct or vector of the invention.

The host cell may be any microbial, prokaryotic or eukaryotic cell, which is suitable for expression of the polypeptide of the invention. Preferably bacterial, yeast, fungal, or mammalian host cells are used. More preferably are species from Escherichia, Saccharomyces, Aspergillus. Even more preferably are strains from Escherichia coli, Saccharomyces cerevisiae, Kluyveromyces lactis, Aspergillus niger which are well-known in the art for overexpressing polypeptides. All cells cited under the section “preferred polypeptides to be used in the screening method and being the object of the invention as such” are also preferred host cells. The host cell may be considered as a recombinant host cell. Alternatively other preferred host cells are insects, CHO cell lines, PER.C6 cells. Suitable procedures for transformation of filamentous fungus may involve a process comprising protoplast formation, transformation of the protoplast, and regeneration of the cell wall in a manner known to the skilled person. Suitable transformation procedures for Aspergillus are described in Yelton et al, 1984, Proceedings of the National Academy of Sciences USA, 81:1470-1474.

According to a preferred embodiment, the host cell hence obtained overexpresses, i.e. produces more than normal amounts of the UDP-galactopyranose mutase polypeptide of the invention and/or exhibits a higher UDP-galactopyranose mutase activity than the parental cell this host cell derives from when both cultured and/or assayed under the same conditions. “Producing more than normal amount” is herein defined as producing more of the polypeptide of the invention than what the parental host cell the transformed host cell derives from will produce when both types of cells (parental and transformed cells) are cultured under the same conditions. Preferably, the host cell of the invention produces at least 3%, 6%, 10% or 15% more of the polypeptide UDP-galactopyranose mutase of the invention than the parental host cell the transformed host cell derives from will produce when both types of cells (parental and transformed cells) are cultured under the same conditions. Also hosts which produce at least 20%, 30%, 40%, 50%, 60%, 70%, 80%, 90%, 100% or 150% more of said polypeptide than the parental cell are preferred. According to another preferred embodiment, the production level of the polypeptide UDP-galactopyranose mutase of the host cell of the invention is compared to the production level of the CBS 120.49 or ATCC9029 strain, which is taken as control. According to an even more preferred embodiment, when the host cell of the invention is an Aspergillus niger strain, the production level of the polypeptide UDP-galactopyranose mutase of the host cell of the invention is compared to the production level of the CBS 120.49 or ATCC9029 strain, which is taken as control.

The assessment of the production level of the polypeptide may be performed at the mRNA level by carrying out a Northern Blot or an array analysis and/or at the polypeptide level by carrying out a Western blot. All these methods are well known to the skilled person. “Exhibiting a higher UDP-galactopyranose mutase activity” is herein defined as exhibiting a higher UDP-galactopyranose mutase activity than the one of the parental host cell the transformed host cell derives from using an assay specific for UDP-galactopyranose mutase activity. Preferably, the assay is the one described by Scherman, which has been already described herein. Preferably, the host cell of the invention exhibits at least 3%, 6%, 10% or 15% higher UDP-galactopyranose mutase activity than the parental host cell the transformed host cell derives from will exhibit as assayed using a specific assay for UDP-galactopyranose mutase assay, which is preferably the Scherman assay. Also host which exhibits at least 20%, 30%, 40%, 50%, 60%, 70%, 80%, 90%, 100% or 150% more of said activity than the parental cell are preferred. According to another preferred embodiment, the level of UDP-galactopyranose mutase activity of the host cell of the invention is compared to the corresponding activity of the CBS 120.49 or ATCC 9029 strain, which is taken as control. According to a more preferred embodiment, when the host cell of the invention is an Aspergillus niger strain, the level of UDP-galactopyranose mutase activity of the host cell of the invention is compared to the corresponding activity of the CBS 120.49 or ATCC 9029 strain, which is taken as control.

The overexpression may have been achieved by conventional methods known in the art, such as by introducing more copies of the UDP-galactopyranose mutase encoding gene into the host, be it on a carrier or in the chromosome, than naturally present. Alternatively, the UDP-galactopyranose mutase encoding gene can be overexpressed by fusing it to highly expressed or strong promoter suitable for high level protein expression in the selected organism, or combination of the two approaches. Alternatively or in combination, a UDP-galactopyranose mutase polypeptide having an enhanced activity as defined earlier can be overexpressed in the host cell of the invention. This can be done using a strong promoter and/or by introducing multiple copies of the encoding gene into the host. The skilled person will know which strong promoter is the most appropriate depending on the identity of the host cell. Preferably when the host cell is an Aspergillus niger strain, the strong promoter is the glucoamylase promoter or the gpdA promoter.

The overexpressing host cell may be used to produce substantial amounts of UDP-galactopyranose mutase which can subsequently be used in the above described method of the invention:
in a first preferred embodiment, the polypeptide of the invention is first produced using the host cell of the invention. Accordingly, in a further aspect, the invention relates to a method for producing the polypeptide of the invention as defined above by culturing the host cell of the invention under suitable culture conditions. Subsequently, the polypeptide produced is contacted in vitro with a potential anti-fungal compound as defined earlier herein (referred to as in vitro method hereafter),
in a second preferred embodiment, the contacting step (step (i)) between the polypeptide of the invention and the potential anti-fungal compound is carried out in vivo in the host cell of the invention as defined earlier herein. Therefore, the polypeptide is contacted with a potential anti-fungal compound in step (i) by virtue of its expression in the host cell of the invention.

### In Vivo Method

Accordingly, the overexpressing host cell according to the invention may itself be used in the method according to the invention (referred to as in vivo method hereafter). Therefore, a screening method for the identification of an anti-fungal compound, which method comprises

(i) contacting an host cell according to the invention, preferably an overexpressing host cell of the invention with a potential anti-fungal compound; and then
(ii) identifying the effect which the potential anti-fungal compound has on the activity of the polypeptide, whereby reduced polypeptide activity is indicative for anti-fungal activity, is also encompassed by the present invention.

The identity of the potential anti-fungal compound and the way the cell and the potential anti-fungal agent are contacted are the same as defined for the former in vitro method.

When cells are used instead of a polypeptide, the skilled person will understand that there are many ways of assessing how the potential anti-fungal compound may affect cell behaviour and/or polypeptide activity. Affecting or reducing polypeptide activity is preferably assessed the way defined earlier.

Preferably, in step (ii) of the in vivo method of the invention, at least one cell wall stress inducing agent is further added and the effect which the potential anti-fungal compound has on the activity of the polypeptide is indicated by a more severe phenotype of the cell associated with the presence of at least one cell wall stress inducing agent than the phenotype of a similar cell treated with the same cell wall stress inducing agent(s) in the absence of the potential anti-fungal compound. The addition of a cell wall stress inducing agent is an additional and/or alternative way of sensoring the potential anti-fungal activity of the potential anti-fungal compound on the polypeptide and/or on the cell of the invention.

The severity of the phenotype of the cell is preferably assessed by measuring its growth ability (measure optical density) and/or by visualising its morphological aspect microscopically. Fungal growth is readily monitored by measuring the optical density in a small container (microliter plate well) using a specific wavelength, preferable between 560 and 620 nm. Microscopical observation reveals morphological abnormalities and defects in growth as well as possible lysis of the fungus.

Several cell wall stress inducing agents are known to the skilled person. Preferably the cell wall stress inducing agent is selected from the group consisting of: calcofluor white (CFW), Congo red, caspofungin, tunicamycin, SDS, and elevated temperature.

The addition of at least one of these mentioned cell wall stress inducing agents is preferably performed as presented in example (in the section secondary screens). More preferably as set out below. 2×10⁴ spores are inoculated in each well of 96-well optical glass bottom microtiter plates in 100 μl 2× Complete Medium and grown for 6 hours at 37° C. The skilled person knows what a complete medium is depending on the identity of the host cell chosen. Preferably, when the host cell is an Aspergillus strain, complete medium comprises the Aspergillus minimum medium as described in Bennett J. W. and Lasure L. L. ((1991), More gene manipulations in fungi. pages 441-447, Academic Press, San Diego) supplemented with 10 g/l yeast extract and 5 g/l casamino acids. After germination of the spores, 100 μl of a two-fold dilution series for each cell wall stress inducing agent is added to individual wells. The effect of each cell wall stress inducing agent is preferably tested for at least three different concentrations, more preferably at least seven different concentrations. After having added the cell wall stress inducing agent, the microtiter plates are incubated for about three more hours at 37° C. After discarding the medium by inverting the microtiter plate, germlings that are adherent to the bottom of each well are observed. Strain ATCC9029 or CBS 120.49 and a dilution series with the compound CFW can be used as negative controls. Light images may be taken on an Axioplan 2 (Zeiss) equipped with a DKC-5000 (Sony) digital photo camera. Preferably, concentrations of caspofungin ranged between 0.4 and 26 μg/ml are used. As for tunicamycin, concentrations ranged between 3 and 176 μg/ml are preferably used. The addition of CFW and/or SDS is preferably performed as described in the example. More preferably, 0.001 till 0.01% w/v SDS and/or 0.01 till 0.1 mg/ml CFW and/or 70 till 500 μg/ml Congo red. Even more preferably, about 0.005% w/v SDS and/or 0.01 till 0.1 mg/ml CFW and/or about 75 μg/ml Congo red is added.

Elevated temperature preferably means as described in the example that the cells are grown either at approximatively 30° C. or at elevated temperature (approximatively 42° C.). If a temperature sensitive growth defect is found as defined below, the addition of an osmotic stabilizer such as 0.5 till 1.5 mM sorbitol is preferably carried out to test the suppressibility of the temperature-growth defect phenotype, which may be indicative of an effective anti-fungal compound. More preferably, about 1.2 M sorbitol is added.

According to a preferred embodiment, a more severe growth phenotype of the cell in the presence of the potential anti-fungal compound resulting in at least 10% less growth compared to the growth of a similar cell treated with the same cell wall stress inducing agent(s) in the absence of the potential anti-fungal compound is indicative for anti-fungal activity of the potential anti-fungal compound.

According to a more preferred embodiment, a more severe growth phenotype of the cell in the presence of the potential anti-fungal compound results in at least 20%, 30%, 40%, 50%, 60%, 80%, 100% less growth.

### Kit

In a further aspect, the invention relates to a kit for carrying out the in vitro screening method of the invention as first defined in the description. The kit comprises in separate containers (i) a polypeptide involved in cell wall synthesis, and (ii) a substrate for said polypeptide. It may further contain markers and controls. Preferably, the polypeptide present in the kit is any one of the preferred polypeptides defined earlier. Accordingly, in a further aspect, the invention relates to the use of this kit for performing the in vitro screening method as first described in the description.

### Anti-Fungal Compound

In yet another aspect, the invention relates to an anti-fungal compound identified by any method of the invention and to a composition comprising an anti-fungal compound of the invention.

Host Cell Producing Less of the Polypeptide of the Invention and/or Exhibiting a Lower UDP-Galactopyranose Mutase Activity

In yet another aspect of the invention, there is provided a host cell, said host cell producing less of the polypeptide of the invention and/or exhibiting a lower UDP-galactopyranose mutase activity than the parental cell this host cell derives from when both cells (parental and host) are cultured and/or assayed under the same conditions.

The identity of the host cell, the polypeptide of the invention are the same as presented earlier herein. The assay for measuring the UDP-galactopyranose mutase was already described herein. Producing less polypeptide and/or exhibiting a lower activity are defined the same way as for cell producing more polypeptide and/or exhibiting a higher activity.

Preferably, the host cell of the invention produces at least 3%, 6%, 10% or 15% less of the polypeptide UDP-galactopyranose mutase of the invention than the parental host cell the transformed host cell derives from will produce when both types of cells (parental and transformed cells) are cultured under the same conditions. Also hosts, which produce at least 20%, 30%, 40%, 50%, 60%, 70%, 80%, 90%, 100% less of said polypeptide than the parental cell are preferred.

According to another preferred embodiment, the production level of the polypeptide UDP-galactopyranose mutase of the host cell of the invention is compared to the production level of the CBS 120.49 or ATCC 9029. According to another preferred embodiment, especially when the host cell of the invention is an Aspergillus niger strain, the production level of the polypeptide UDP-galactopyranose mutase of the host cell of the invention is compared to the production level of the CBS 120.49 or ATCC 9029.

The assessment of the production level of the polypeptide may be performed at the mRNA level by carrying out a Northern Blot or an array analysis and/or at the polypeptide level by carrying out a Western blot. All these methods are well known to the skilled person.

Preferably, the host cell of the invention exhibits at least 3%, 6%, 10% or 15% lower UDP-galactopyranose mutase activity than the parental host cell the transformed host cell derives from will exhibits as assayed using a specific assay for UDP-galactopyranose mutase assay, which is preferably the Scherman assay. Also hosts, which exhibit at least 20%, 30%, 40%, 50%, 60%, 70%, 80%, 90%, or 100% less of said activity than the parental cell are preferred. According to another preferred embodiment, the polypeptide UDP-galactopyranose mutase activity of the host cell of the invention is compared to the corresponding activity of the CBS 120.49 or ATCC 9029.

According to another preferred embodiment, especially when the host cell of the invention is an Aspergillus niger strain, the polypeptide UDP-galactopyranose mutase activity of the host cell of the invention is compared to the corresponding activity of the CBS 120.49 or ATCC 9029.

According to a more preferred embodiment, the host cell does not produce any detectable amounts of the polypeptide of the invention and/or does not exhibit any detectable UDP-galactopyranose mutase activity. Preferably, the host cell does not produce or produces substantially no UDP-galactopyranose mutase.

Alternatively, according to another more preferred embodiment, the host cell produces an inducible amount of the polypeptide of the invention and/or exhibit an inducible UDP-galactopyranose mutase activity.

The lowering of the expression level of the polypeptide of the invention and/or the lowering of its activity level may have been achieved by conventional methods known in the art, such as by inactivating or down-regulating the endogenous UDP-galactopyranose mutase encoding gene of the host. This inactivation or down regulation may have been achieved by deletion of one or more nucleotides in the encoding gene. In another embodiment, the invention relates to a host, preferably a filamentous fungus which has a mutation in its UDP-galactopyranose mutase encoding gene. Preferably to construct a host having an inactivated UDP-galactopyranose mutase gene, a replacement or inactivation vector is prepared and is subsequently introduced into the host by transformation. The skilled person will know how to construct such a vector.

Alternatively or in combination with the inactivation of the endogenous gene, the expression of the UDP-galactopyranose mutase gene can be lowered by fusing it to a weak promoter suitable for low level protein expression in the selected organism. Preferably when the host cell is an Aspergillus niger strain, a weak promoter is the trpC promoter of Aspergillus nidulans or the pyrG promoter of Aspergillus niger.

Alternatively or in combination with the inactivation of the endogenous gene, the expression of the UDP-galactopyranose mutase gene can be rendered inducible by fusing it to an inducible promoter suitable for inducible level protein expression in the selected organism. Preferably when the host cell is an Aspergillus niger strain, the inducible promoter is the glucoamylase promoter, which can be inducing by starch (Fowler T, et al., 1990. Regulation of the glaA gene of Aspergillus niger. Curr Genet. 18:537-545) or the inuE promoter, which can be induced by sucrose (Moriyama S et al., 2003. Molecular cloning and characterization of an exoinulinase gene from Aspergillus niger strain 12 and its expression in Pichia pastoris. J Biosci Bioeng. 96:324-331).

In a further aspect, the invention relates to the use of this host cell producing less of the polypeptide of the invention and/or exhibiting a lower UDP-galactopyranose mutase activity than the parental cell this host cell derives from when both cells (parental and host) are cultured under the same conditions. This host cell is attractive for producing a polypeptide of interest. Any polypeptide may be produced using this host. Preferably, glycoproteins are produced. This host cell is expected to produce glycoproteins, which will have less galactomannan and/or galactofuranose sugars than identical glycoproteins produced by similar host cells still expressing an UDP-galactopyranose mutase polypeptide. Preferably, the glycoprotein produced has substantially no galactomannan and/or galactofuranose sugars. These sugars are main components of the fungal cell wall polysaccharide, but are not present in mammal glycopolypeptides. These glycoproteins hence produced are expected not to give any major allergenic reaction of the mammal host these glycoproteins would be administered to.

The present invention is further described by the following examples which should not be construed as limiting the scope of the invention.

### EXAMPLES

A reporter strain was constructed in which the agsA promoter was fused both to the acetamidase (amdS) selection marker and to the nuclear targeted GFP (H2B-GFP) reporter construct as described in WO 03/20922, allowing the selection for trans-acting mutations that activate the cell wall integrity response and thus give a constitutively increased agsA promoter activity. The primary screen yielded 240 mia mutants (Mutant with induced agsA promoter activity) that were subjected to various secondary screens (e.g. osmotic remediable temperature sensitivity, and Calcofluor White-, and SDS-sensitivity). Complementation analysis showed that the miaA, miaB and miaC mutants were complemented by cosmids with overlapping inserts indicating that their mutations are possibly allelic.

### Strains, Transformations, and Growth Conditions

A. niger N402 (a cspA1 derivative of ATCC9029, Bos et al., 1988) and AB4.1 (van Hartingsveldt et al., 1987) a pyrG⁻ derivative of N402 were used in this study. Strains were grown on minimal medium (MM) (Bennett and Lasure, 1991) containing 1% (w v⁻¹) glucose and 0.1% (w v⁻¹) casamino acids or on complete medium (CM), containing 0.5% (w v⁻¹) yeast extract in addition to MM. When required, plates were supplemented with uridine (10 mM) or hygromycin (100 μg ml⁻¹). Conidia were isolated with 0.9% (w v⁻¹) NaCl from CM plates after growth for 4-6 days at 30° C. MM agar plates containing acetamide as a sole nitrogen source were made as described (Kelly and Hynes, 1985). Transformation of A. niger was performed as described by Punt and van den Hondel (1992), using 40 mg lysing enzymes (Sigma, L-1412) per g fresh weight mycelium. For co-transformations using the hygromycin selection marker, pAN7-1 (accession number Z32698) was used. Escherichia coli strain DH5α (Invitrogen) was transformed by electroporation, according to the suppliers manual, for the propagation and amplification of cosmids. XL1-Blue (Stratagene, La Jolla, Calif.) was transformed using the heat shock protocol as described by Inoue et al. (1990) and used for the amplification of plasmids. E. coli was grown in LB as described in Sambrook et al. (1989), with the addition of 50 μg ml⁻¹ ampicillin when required.

### Molecular Techniques

Fungal chromosomal DNA was isolated as described by Kolar et al. (1988). [α-³²P]dCTP labeled probes were synthesized using the Rediprime II DNA labeling system (Amersham Pharmacia Biotech) according to the instructions of the manufacturer. Southern blot analyses were performed as described by Sambrook et al. (1989) using HybondN+ (Amersham Pharmacia Biotech), and hybridization signals were detected using a Phosphor Imager (Molecular Dynamics). Restriction enzymes were obtained from Invitrogen and used according to the supplier's manual. The ligation of DNA fragments was performed with the Rapid DNA ligation kit (Roche). When required, fragments were dephosphorylated with Shrimp Alkaline Phosphatase (Roche). Sequencing was performed by ServiceXS (Leiden, Netherlands).

### Mutagenesis and the Primary Mutant Screen

The strains used for mutagenesis were constructed as follows. The AB4.1 (pyrG⁻) strain was transformed with PagsA-amdS-TamdS-pyrG* or PagsA-H2B-GFP-TtrpC-pyrG* (WO03020922). For both constructs, transformants were selected that had a single copy of the construct integrated on the pyrG locus based on Southern analysis (data not shown), and were named RD1.7 and RD5.43 respectively. Next, strain RD1.7 was co-transformed with PagsA-H2B-GFP-TtrpC and strain RD5.43 was co-transformed with PagsA-amdS-TamdS, using pAN7-1 in both cases. Transformants were analysed by PCR for the presence of the complete reporter constructs, yielding strains RD6.13 and RD6.47 (containing PagsA-amdS-TamdS targeted to the pyrG locus and PagsA-H2B-GFP-TtrpC co-transformed) and strains RD15.4 and RD15.8 (containing PagsA-H2B-GFP-TtrpC targeted to the pyrG locus and PagsA-amdS-TamdS co-transformed). All four strains were subjected to UV mutagenesis. Freshly harvested spores were diluted to 1×10⁷ spores ml⁻¹ and 15 ml spore solutions were mutagenised in a Bio-Rad cross linker (maximum energy output at λ=254 nm, UV dose 60 J s⁻¹ m²) for 0-100 seconds with 10 seconds intervals. Survival rates at the different time-points were determined by plating out dilutions of the mutagenised conidia suspensions on CM-plates. The conidia from spore suspensions with a ˜66% survival rate were used for the primary screen. For each of the four strains, 60 MM-plates with acetamide as the sole nitrogen source were inoculated with 1×10⁴ conidia and incubated at 30° C. After five days a single fast growing colony from each plate was transferred to CM-plates and purified two times, yielding 240 primary mutants.

### Secondary Screens

Growth on acetamide. The purified mutants were re-tested for their ability to grow on acetamide plates at 30° C. Equal amounts of conidia (˜5×10²) were spotted on MM-plates, with acetamide as the sole nitrogen source and images were taken after 3 days.

Nuclear GFP levels. For microscopical images, conidia were grown on coverslips in MM with casamino acids at 30° C. for 18 hours. The coverslips with adherent conidia were placed on microscope slides and microscopic GFP images were taken on an Axioplan 2 (Zeiss) equipped with a DKC-5000 (Sony) digital photo camera using a fixed exposure time of one second. As a negative control N402, as a positive control PgpdA-H2B-GFP (+), and for the basal level the non-mutagenised parental strains (PagsA-H2B-GFP) were used. GFP images were analysed using Qwin Pro (LEICA, v2.2) In brief, the green channels of the images were analysed by selecting all green pixels with a value >130, which corresponded as expected to the nuclei. The average GFP values (Mean Gm) and the maximum GFP values (Max Gm) were determined for these selections and compared to the non-mutagenised values. Mutants in which the average or maximum GFP values were higher when compared to non-mutagenised strains were scored as mutants with increased GFP expression from the agsA promoter.

Temperature sensitivity. Ten fold dilutions of spores from the parental and the mutant strains starting with 1×10⁴ spores were grown at 30° C., 37° C. or 42° C. on MM-plates for four days. The colony morphology (e.g. sporulation and diameter) was compared between the two plates.

Osmotic remediability at 30° C. and 42° C. The effect of the addition of the osmotic stabilizer sorbitol was examined. Therefore strains were grown as described above on MM with or without the addition of 1.2 M of sorbitol at 30° C. or 42° C. for four days.

Sensitivity towards SDS Congo red and CFW. MM plates containing 0.005% (w v⁻¹) SDS, 75 μg/ml Congo red, 0.1 mg ml⁻¹ CFW or 0.01 mg ml⁻¹ CFW were inoculated with the spores of the parental and mutant strains and grown at 30° C. 37° C., 42° C. for four days. Colony size and morphology were compared to MM-plates without additives grown under the same conditions.

### Complementation of the Cell Wall Mutants

PyrG⁻ derivatives of miaA, miaB and miaC mutants were obtained by plating out 1×10⁵ spores on MM-5-FAO-plates (0.75 g l⁻¹ 5-fluoro-orotic acid (5-FOA, USBiological) in which the NaNO₃ was replaced by 10 mM prolin as N-source. Additionally, the medium further contains 10 mM uridine. Conidiating colonies were purified twice on MM plates supplemented with 10 mM uridine and analysed based on their phenotype on MM-plates containing 0.005% (w v⁻¹) SDS with and without uridine. Hence, mutations in both pyrE, an orotate phosphoribosyl transferase (OPRTase; EC 2.4.2.10) and pyrG, an orotidine-5′-monophosphate decarboxylase (OMPdecase; EC 4.1.1.23) can confer resistance towards 5-FOA (Boeke et al., 1984, Takeno et al., 2004), the resistant strains were transformed with plasmids containing either pyrE (pMApyrE) or pyrG (pAB4.1) and analysed for complementation based on growth without uridine. 5-FOA resistant strains that could only be complemented by pyrG were used for further analysis. The sequence of pyrE was submitted to DDBJ/EMBL/GenBank databases with accession number AY840014.

The pyrG⁻ mutants were transformed with a genomic cosmid library (kindly provided by Dr. F. Schuren and Dr. P. Punt, TNO Nutrition, The Netherlands). Transformants were selected on transformation plates based on the ability to grow without uridine (pyrG complementation). Complementation of the mutant phenotype was analysed by screening for strains that had obtained the parental SDS sensitivity at 42° C. After transformation with the genomic cosmid library, spores were isolated from transformation plates, transferred to plates containing minimal medium with 0.005% (w v⁻¹) SDS, and grown for four days at 42° C. Cosmids from the putative complemented A. niger strains were isolated using the protocol for isolation of genomic DNA (Kolar et al., 1988). The cosmids were transformed to E. coli (DH5α) and grown on LB plates with ampicillin. Subsequent cosmid isolations from 40 ml of overnight cultures were performed using small scale DNA isolation method as described by Sambrook et al. (1989). After amplification in E. coli, the cosmids were subjected to restriction analysis. Of each independently obtained complemented transformant, the restriction pattern of at least four cosmids was obtained. Non-identical cosmids were transformed back to their corresponding mia (pyrG⁻) strain and analysed for their ability to complement the mutation, based on restoring the wild-type temperature-sensitivity and SDS-sensitivity. Based on restriction pattern and the complementation test, a single complementing cosmid was found for miaA. Three different, but overlapping cosmids were obtained that complemented the miaB mutant. The restriction pattern of miaA and miaB were similar indicating that they might be overlapping. Four complementing cosmids were obtained for miaC. Unfortunately these miaC cosmids were resistant to restriction enzyme digestions and end-sequencing (see below) which hampered the further analysis. The possible overlap found by restriction analyses of miaA and miaB complementing cosmids was confirmed by sequencing of the ends of the insert of the cosmids. Primers cosT7 and cosUL were used for sequencing the ends of the inserts. Comparison of the cosmids to the A. niger genome sequence and to each other showed that the miaA complementing cosmid and the three miaB complementing cosmids shared a 35 kb region containing at least 9 predicted ORFs. Further subcloning and complementation analysis pointed to two candidate ORFs (8650 and 8660). These ORF were PCR amplified from genomic DNA of the wild type strain (N402) using primer pair 8650P1/8650P2 and primer pair 8660P3/8660P2 respectively. PCR fragments for both genes were transformed to miaA, miaB and miaC mutants. Only the PCR fragments with the 86600RF complemented all the phenotypes of the mutants (Temperature sensitive grow defect at 42° C., the SDS and CFW-hypersensitivity). Thus, the identification of the gene(s) complementing the miaA, miaB and miaC mutants was achieved and appeared to be 8660. The protein displays strong sequence similarity to the UDP-galactopyranose mutase encoding gene and we will refer to this gene as glfA. The genomic sequence is given in SEQ ID NO: 3 and the deduced cDNA and protein sequences are given in SEQ ID NO: 2 and 1 respectively.

Sequencing of the glfA alleles of the miaA and miaC mutants confirmed mutations in the gene encoding GlfA. The glfA locus in the miaA strain contains a point mutation (T to C) at position 1756 (A nucleotide of the start codon ATG=1), resulting in an amino acid change at position 462 (F to S). The glfA locus in the miaC strain contains two mutations at position 725 and 727, (both T to C mutations) resulting in two amino acid changes at position 157 and 158 (L to P and F to L respectively).

Construction of a Strain Lacking glfA

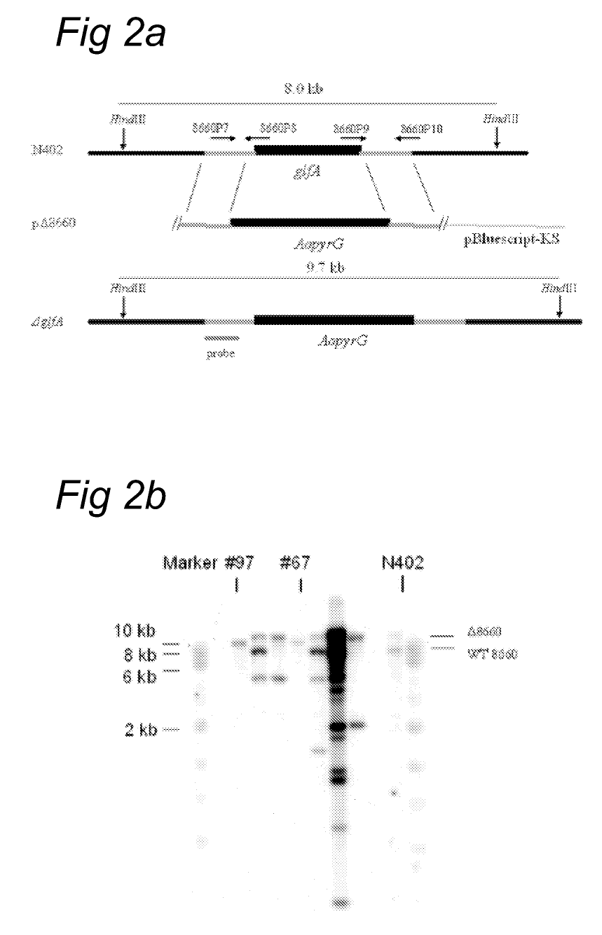

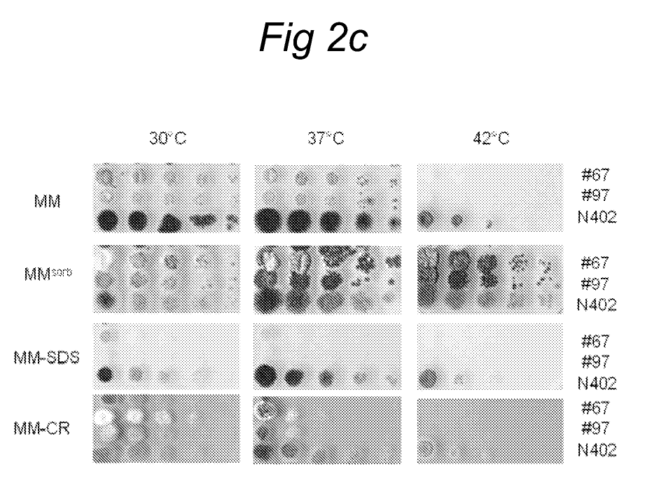

A deletion mutant of the complete glfA gene was constructed. To this end, a plasmid was constructed which contained the pyrG selection marker of A. oryzae flanked by 5′ and 3′ flanking regions of the glfA gene (FIG. 2A). The construct was made as follows: using primers 8660P7 and 8660P8 (see table 1 below), a 1.2 kb fragment containing the 5′ flank of glfA, was amplified, digested with XbaI and NotI and subsequently cloned into XbaI and NotI digested pBluescript-KS. The construct construct p5-8660 was hence obtained. Primers 8660P9 en 8660P10 were used to amplify the 1.0 kb 3′ flank of the glfA locus. The fragment was digested with XbaI and NotI and cloned into XbaI and EcoRI digested pBluescript-KS fragment to give p3-8660. The A. oryzae pyrG gene was isolated as a 3.4 kb Xba fragment from pAO4-13 (de Ruiter-Jacobs et al., 1989). p5-8660 was opened with XbaI and EcoRI and the AopyrG fragment and XbaI-EcoRI fragment from p3-8660 were cloned into this plasmid to give pΔ8660. This plasmid was subsequently linearized with NotI and transformed to AB4.1. Primary transformants were purified twice on MM lacking uridine. A small number (6 of the 140) of transformants grow poorly at high temperatures. These mutants and some others were subjected to Southern blot analysis. Deletion of the 8660 locus by homologous recombination was expected to result in appearance of a 9.7 kb fragment after digestion with HindIII and the loss of a 8.0 kb hybridising fragment which was expected in the wild type strain. The Southern blot in FIG. 2B confirms that the glfA gene had been deleted in strain #67 and #97. The knockouts were further analysed for temperature sensitive growth, suppression of temperature sensitive growth by high osmolarity condition, hypersensitivity towards SDS and Congo Red. (FIG. 2C) Comparison of the growth phenotypes of the miaA, miaB and the Δ8660 strains (temperature sensitive growth defect and Congo Red sensitivity) indicate that the miaA and miaB mutants were as severe as the knockout strains (not shown). The phenotypes of the Δ8660 strain was again complemented by retransformation of the PCR fragment (obtained with primers 8660P3 and 8660P2) containing the 8660 gene with promoter and terminator sequences (not shown).

TABLE 1
list of primers used

| primer name | | restriction |
|---|---|---|
| SEQ ID NO | primer sequence 5′→3′ | enzyme |
| cosT7 | GCTTATCGATGATAAGCGGTC | |
| SEQ ID NO:4 | | |
| CosUL | AAAGCTTGCATGCCTGCAGGTCG | |
| SEQ ID NO:5 | | |
| 8650P1 | CCAGGCGCAGTAAAGGGTT | — |
| SEQ ID NO:6 | | |
| 8650P2 | CATACGCCAT GCAGGGCT | — |
| SEQ ID NO:7 | | |
| 8660P2rev | CATCGCTGTCAATGAGCCAA | — |
| SEQ ID NO:8 | | |
| 8660P3for | CATTGCCGTTCTTGGTAATCG | — |
| SEQ ID NO:9 | | |
| 8660P7for | ATAAGAATGCGGCCGCTTGATGAAGGT | NotI |
| SEQ ID NO:10 | GATCGGGCT | |
| 8660P8rev | GCTCTAGATGTGAAGCTGCAGGGACAG | XbaI |
| SEQ ID NO:11 | TA | |
| 8660P9for | GCTCTAGAGTGGTATCTGGATTGCCGT | XbaI |
| SEQ ID NO:12 | C | |
| 8660P10rev | CGGAATTCTGATGAAATATGGCTCCCG | EcoRI |
| SEQ ID NO:13 | TT | |

Production and Analysis of the Mammalian Tissue Plasminogen Activator (t-PA) Using the Strain which Lacks the glfA Gene.

An expression vector for the production of t-PA was prepared: pgpdA-Tpa was constructed and isolated as described in Wiebe M G, et al (Wiebe M G, et al Production of tissue plasminogen activator (t-PA) in Aspergillus niger. Biotechnol Bioeng. 2001 September; 76(2):164-74).

The A. niger N402 and A. niger glfA deletion strain #67 as obtained earlier were transformed with both pgpdA-Tpa and pAN7-1. as described by Punt and van den Hondel (1992). Transformants were analyzed by PCR for the presence of the vector pgpdA-Tpa. Two transformants of both strains containing pgpdA-Tpa were grown as described by Wiebe et al. (2001) and partially purified t-PA was obtained from the supernatants as described by Wiebe et al. (2001). Extracellular glucoamylase was purified from the supernatants as described by Teotia S et al (Teotia S et al One-step purification of glucoamylase by affinity precipitation with alginate. J Mol. Recognit. 2001 September-October; 14(5):295-9).

The N-linked glycans to t-PA and glucoamylase were analyzed as described for alpha-galactosidase by Wallis G L et al. (Wallis G L et al, Galactofuranoic-oligomannose N-linked glycans of alpha-galactosidase A from Aspergillus niger. Eur J. Biochem. 2001 August; 268(15):4134-43). Glycoproteins obtained from mycelial cell wall were analyzed as for the ones of A. fumigatus by Leitao E A et al (Leitao E A, et al, Beta-galactofuranose-containing O-linked oligosaccharides present in the cell wall peptidogalactomannan of Aspergillus fumigatus contain immunodominant epitopes. Glycobiology. 2003 October; 13(10):681-92.0.

The analysis showed that the N-linked glycans obtained from t-PA and glucoamylase produced by the A. niger glfA deletion strain #67 contained at least 20% less Galf than the corresponding N-linked glycans obtained from the proteins produced by the A. niger N402 strain.

### REFERENCE LIST

Bennett, J. W. and Lasure, L. L. (1991). More Gene Manipulations in Fungi, pp. 441-447, Academic Press, San Diego.
Bickle, M., Delley, P. A., Schmidt, A., and Hall, M. N. (1998). Cell wall integrity modulates RHO1 activity via the exchange factor ROM2. EMBO J. 17, 2235-2245.
Boeke, J. D., LaCroute, F., and Fink, G. R. (1984). A positive selection for mutants lacking orotidine-5′-phosphate decarboxylase activity in yeast: 5-fluoro-orotic acid resistance. Mol. Gen. Genet. 197, 345-346.
Boorsma, A., de Nobel, H., ter Riet, B., Bargmann, B., Brul, S., Hellingwerf, K. J., and Klis, F. M. (2004). Characterization of the transcriptional response to cell wall stress in Saccharomyces cerevisiae. Yeast 21, 413-427.
Borgia, P. T. and Dodge, C. L. (1992). Characterization of Aspergillus nidulans mutants deficient in cell wall chitin or glucan. J. Bacteriol. 174, 377-383.
Bos, C. J., Debets, A. J., Swart, K., Huybers, A., Kobus, G., and Slakhorst, S. M. (1988). Genetic analysis and the construction of master strains for assignment of genes to six linkage groups in Aspergillus niger Curr. Genet. 14, 437-443.
Corrick, C. M., Twomey, A. P., and Hynes, M. J. (1987). The nucleotide sequence of the amdS gene of Aspergillus nidulans and the molecular characterization of 5′ mutations. Gene 53, 63-71.
Costigan, C., Gehrung, S., and Snyder, M. (1992). A synthetic lethal screen identifies SLK1, a novel protein kinase homolog implicated in yeast cell morphogenesis and cell growth. Mol. Cell. Biol. 12, 1162-1178.
Dailies, N., Francois, J., and Paquet, V. (1998). A new method for quantitative determination of polysaccharides in the yeast cell wall. Application to the cell wall defective mutants of Saccharomyces cerevisiae. Yeast 14, 1297-1306.
Damveld, R. A., Vankuyk, P. A., Arentshorst, M., Klis, F. M., van den Hondel, C. A. M. J. J., and Ram, A. F. J. (2005). Expression of agsA, one of five 1,3-α-D-glucan synthase-encoding genes in Aspergillus niger, is induced in response to cell wall stress. Fung. Genet. Biol. 42, 165-177.
de Groot, P. W. J., Ruiz, C., Vazquez de Aldana, C. R., Dueòas, E., Cld, V. J., Del Rey, F., Rodriquez-Peña, J. M., Pérez, P., Andel, A., Caubin, J., Arroyo, J., Garcia, J. C., Gil, C., Molina, M., Garcia, L. J., Nombela, C., and Klis, F. M. (2001). A genomic approach for the identification and classification of genes involved in cell wall formation and its regulation in Saccharomyces cerevisiae. Comp. Funct. Genom. 2, 124-142.
de Nobel, H., Lawrie, L., Brul, S., Klis, F., Davis, M., Alloush, H., and Coote, P. (2001). Parallel and comparative analysis of the proteome and transcriptome of sorbic acid-stressed Saccharomyces cerevisiae. Yeast 18, 1413-1428.
Garcia, R., Bermejo, C., Grau, C., Perez, R., Rodriguez-Pena, J. M., Francois, J., Nombela, C., and Arroyo, J. (2004). The global transcriptional response to transient cell wall damage in Saccharomyces cerevisiae and its regulation by the cell integrity signaling pathway. J. Biol. Chem. 279, 15183-15195.
Gouka, R. J., Hessing, J. G., Stam, H., Musters, W., and van den Hondel, C. A. (1995). A novel strategy for the isolation of defined pyrG mutants and the development of a site-specific integration system for Aspergillus awamori. Curr. Genet. 27, 536-540.
Gustin, M. C., Albertyn, J., Alexander, M., and Davenport, K. (1998). MAP kinase pathways in the yeast Saccharomyces cerevisiae. Microbiol. Mol. Biol. Rev. 62, 1264-1300.
Hanegraaf, P. P., Punt, P. J., van den Hondel, C. A., Dekker, J., Yap, W., van Verseveld, H. W., and Stouthamer, A. H. (1991). Construction and physiological characterization of glyceraldehyde-3-phosphate dehydrogenase overproducing transformants of Aspergillus nidulans. Appl. Microbiol. Biotechnol. 34, 765-771.
Heinisch, J. J., Lorberg, A., Schmitz, H. P., and Jacoby, J. J. (1999). The protein kinase C-mediated MAP kinase pathway involved in the maintenance of cellular integrity in Saccharomyces cerevisiae. Mol. Microbiol. 32, 671-680.
Igual, J. C., Johnson, A. L., and Johnston, L. H. (1996). Coordinated regulation of gene expression by the cell cycle transcription factor Swi4p and the protein kinase C MAP kinase pathway for yeast cell integrity. EMBO J. 15, 5001-5013.
Inoue, H., Nojima, H., and Okayama, H. (1990). High efficiency transformation of Escherichia coli with plasmids. Gene 96, 23-28.
Irie, K., Takase, M., Lee, K. S., Levin, D. E., Araki, H., Matsumoto, K., and Oshima, Y. (1993). MKK1 and MKK2, which encode Saccharomyces cerevisiae mitogen-activated protein kinase-kinase homologs, function in the pathway mediated by protein kinase C. Mol. Cell Biol. 13, 3076-3083.
Kelly, J. M. and Hynes, M. J. (1985). Transformation of Aspergillus niger by the amdS gene of Aspergillus nidulans. EMBO J. 4, 475-479.
Kolar, M., Punt, P. J., van den Hondel, C. A., and Schwab, H. (1988). Transformation of Penicillium chrysogenum using dominant selection markers and expression of an Escherichia coli lacZ fusion gene. Gene 62, 127-134.
Lagorce, A., Berre-Anton, V., Aguilar-Uscanga, B., Martin-Yken, H., Dagkessamanskaia, A., and Francois, J. (2002). Involvement of GFA1, which encodes glutamine-fructose-6-phosphate amidotransferase, in the activation of the chitin synthesis pathway in response to cell-wall defects in Saccharomyces cerevisiae. Eur. J. Biochem. 269, 1697-1707.
Lagorce, A., Hauser, N. C., Labourdette, D., Rodriguez, C., Martin-Yken, H., Arroyo, J., Hoheisel, J. D., and Francois, J. (2003). Genome-wide analysis of the response to cell wall mutations in the yeast Saccharomyces cerevisiae. J. Biol. Chem. 278, 20345-20357.
Lee, K. S., Irie, K., Gotoh, Y., Watanabe, Y., Araki, H., Nishida, E., Matsumoto, K., and Levin, D. E. (1993). A yeast mitogen-activated protein kinase homolog (Mpk1p) mediates signalling by protein kinase C. Mol. Cell. Biol. 13, 3067-3075.
Lussier, M., White, A. M., Sheraton, J., di Paolo, T., Treadwell, J., Southard, S. B., Horenstein, C. I., Chen-Weiner, J., Ram, A. F., Kapteyn, J. C., Roemer, T. W., Vo, D. H., Bondoc, D. C., Hall, J., Zhong, W. W., Sdicu, A. M., Davies, J., Klis, F. M., Robbins, P. W., and Bussey, H. (1997). Large scale identification of genes involved in cell surface biosynthesis and architecture in Saccharomyces cerevisiae. Genetics 147, 435-450.
Maruyama, J., Nakajima, H., and Kitamoto, K. (2001). Visualization of nuclei in Aspergillus oryzae with EGFP and analysis of the number of nuclei in each conidium by FACS. Biosci. Biotechnol. Biochem. 65, 1504-1510.
Mizutani, O., Nojima, A., Yamamoto, M., Furukawa, K., Fujioka, T., Yamagata, Y., Abe, K., and Nakajima, T. (2004). Disordered cell integrity signaling caused by disruption of the kexB gene in Aspergillus oryzae. Eukaryot Cell. 3, 1036-1048.
Osmond, B. C., Specht, C. A., and Robbins, P. W. (1999). Chitin synthase III: synthetic lethal mutants and “stress related” chitin synthesis that bypasses the CSD3/CHS6 localization pathway. Proc. Natl. Acad. Sci. U.S.A. 96, 11206-11210.
Paravicini, G., Cooper, M., Friedli, L., Smith, D. J., Carpentier, J. L., Klig, L. S., and Payton, M. A. (1992). The osmotic integrity of the yeast cell requires a functional PKC1 gene product. Mol. Cell. Biol. 12, 4896-4905.
Popolo, L., Gilardelli, D., Bonfante, P., and Vai, M. (1997). Increase in chitin as an essential response to defects in assembly of cell wall polymers in the ggp1delta mutant of Saccharomyces cerevisiae. J. Bacteriol. 179, 463-469.
Punt, P. J. and van den Hondel, C. A. (1992). Transformation of filamentous fungi based on hygromycin B and phleomycin resistance markers. Methods Enzymol. 216, 447-457.
Ram, A. F., Wolters, A., ten Hoopen, R., and Klis, F. M. (1994). A new approach for isolating cell wall mutants in Saccharomyces cerevisiae by screening for hypersensitivity to calcofluor white. Yeast 10, 1019-1030.
Ram, A. F., Arentshorst, M., Damveld, R. A., vankuyk, P. A., Klis, F. M., and van den Hondel, C. A. (2004). The cell wall stress response in Aspergillus niger involves increased expression of the glutamine:fructose-6-phosphate amidotransferase-encoding gene (gfaA) and increased deposition of chitin in the cell wall. Microbiology 150, 3315-3326.
Sambrook, J., Fritsch, E. F., and Maniatis, T. (1989). Molecular Cloning: a Laboratory Manual, Cold Spring Harbor Laboratory Press, Plainview N.Y.
Shimizu, J., Yoda, K., and Yamasaki, M. (1994). The hypo-osmolarity-sensitive phenotype of the Saccharomyces cerevisiae hpo2 mutant is due to a mutation in PKC1, which regulates expression of beta-glucanase. Mol. Gen. Genet. 242, 641-648.
Takeno, S., Sakuradani, E., Murata, S., Inohara-Ochiai, M., Kawashima, H., Ashikari, T., and Shimizu, S. (2004). Cloning and sequencing of the ura3 and ura5 genes, and isolation and characterization of uracil auxotrophs of the fungus Mortierella alpina 1S-4. Biosci. Biotechnol Biochem. 68, 277-285.
Terashima, H., Yabuki, N., Arisawa, M., Hamada, K., and Kitada, K. (2000). Up-regulation of genes encoding glycosylphosphatidylinositol (GPI)-attached proteins in response to cell wall damage caused by disruption of FKS1 in Saccharomyces cerevisiae. Mol. Gen. Genet. 264, 64-74.
Valdivia, R. H. and Schekman, R. (2003). The yeasts Rho1p and Pkc1p regulate the transport of chitin synthase III (Chs3p) from internal stores to the plasma membrane. Proc. Natl. Acad. Sci. U.S.A. 100, 10287-10292.
van Hartingsveldt, W., Mattern, I. E., van Zeijl, C. M., Pouwels, P. H., and van den Hondel, C. A. (1987). Development of a homologous transformation system for Aspergillus niger based on the pyrG gene. Mol. Gen. Genet. 206, 71-75.
Verdoes, J. C., Punt, P. J., Schrickx, J. M., van Verseveld, H. W., Stouthamer, A. H., and van den Hondel, C. A. (1993). Glucoamylase overexpression in Aspergillus niger molecular genetic analysis of strains containing multiple copies of the glaA gene. Transgenic es. 2 84-92.

1-31. (canceled)

32. A polypeptide having UDP-galactopyranose mutase activity, wherein the amino acid sequence of the polypeptide is at least 48% identical with SEQ ID NO:1.

33. The polypeptide according to claim 32, the amino acid sequence of which is SEQ ID NO:1.

34. The polypeptide according to claim 32, obtained from a fungus.

35. The polypeptide according to claim 34 wherein the fungus is a filamentous fungus.

36. The polypeptide according to claim 35, wherein the filamentous fungus is Aspergillus niger.

37. A kit comprising in separate containers:
(a) a polypeptide with enzymatic activity according to claim 32; and,
(b) a substrate for said polypeptide.

38. A screening method for the identification of an anti-fungal compound, which method comprises:
(a) contacting a candidate anti-fungal compound with a polypeptide which is a UDP-galactopyranose mutase enzyme involved in cell wall synthesis; and subsequently,
(b) identifying the effect of the candidate anti-fungal compound on the activity of the polypeptide;
wherein reduced polypeptide activity is indicative of anti-fungal activity of the candidate anti-fungal compound.

39. The screening method according to claim 38, wherein the polypeptide has an amino acid sequence which has at least about 48% identity with SEQ ID NO:1.

40. The screening method according to claim 38, wherein the activity of the polypeptide is enzymatic activity.

41. The screening method according to claim 39, wherein the enzymatic activity comprises conversion of UDP-galactopyranose into UDP-galactofuranose.

42. The screening method according to claim 38, wherein a reduction of the activity of the polypeptide in the presence of the candidate anti-fungal compound of at least 20% compared to the activity of the polypeptide in the absence of said candidate anti-fungal compound is indicative of said anti-fungal activity of the candidate anti-fungal compound.

43. The screening method according to claim 38, wherein the activity of the polypeptide is determined using (i) a microplate assay that measures radioactivity or fluorescence or (ii) HPLC.

44. A nucleic acid encoding the polypeptide of claim 32.

45. The nucleic acid according to claim 44, the sequence of which a has at least 50% identity with SEQ ID NO:2.

46. The nucleic acid according to claim 45 that has one or more of the following properties:
(a) encodes the same amino acid sequence as does the nucleic acid of claim 45 on the basis of (1) redundancy of the genetic code (2) a silent mutation or (3) a nucleotide substitution;
(b) is an allelic variant of SEQ ID NO:2 present in the organism from which the nucleic acid is obtained; or
(c) encodes a variant polypeptide of the polypeptide encoded by the nucleic acid of claim 45, which variant is:
(i) a conservative substitution variant, or
(ii) a fragment,
which substitution variant or fragment has said UDP-galactopyranose mutase enzymatic activity.

47. The nucleic acid according to claim 44 the nucleotide sequence of which is SEQ ID NO:2.

48. A nucleic acid construct comprising the nucleic acid of claim 44.

49. An expression vector comprising the nucleic acid construct of claim 48.

50. A host cell comprising the nucleic acid construct of claim 48.

51. The host cell according to claim 50, wherein the host cell produces more of the polypeptide and/or exhibits higher UDP-galactopyranose mutase activity than a parental cell from which said host cell is derived when the parental cell and the host cell are cultured and/or assayed under the same conditions.

52. The host cell according to claim 50 that
(i) produces at least 3% less of the polypeptide having UDP-galactopyranose mutase enzymatic activity, wherein the amino acid sequence of the polypeptide is at least 48% identical with SEQ ID NO:1, and/or
(ii) exhibits at least 3% lower UDP-galactopyranose mutase enzymatic activity, as compared to a parental cell from which the host cell is derived when the parental cell and the host cell are cultured and/or assayed under the same conditions.

53. The host cell according to claim 52 that does not produce detectable amounts of the polypeptide and/or does not exhibit detectable UDP-galactopyranose mutase enzymatic activity.

54. The host cell according to claim 52, wherein the polypeptide and/or said UDP-galactopyranose mutase activity is inducible in the host cell.

55. A screening method for the identification of an anti-fungal compound, which method comprises:
(a) contacting a candidate anti-fungal compound with host cells according to claim 50 that express said polypeptide; and subsequently,
(b) identifying the effect of the candidate anti-fungal compound on the activity of the polypeptide;
wherein reduced activity of said polypeptide is indicative of anti-fungal activity of the candidate anti-fungal compound.

56. The screening method according to claim 55, wherein the polypeptide is a fungal UDP-galactopyranose mutase enzyme.

57. The screening method according to claim 55, wherein in step (b) at least one cell wall stress inducing agent or condition is added or applied to said host cells and said candidate compound,
wherein the effect of the candidate anti-fungal compound on the activity of the polypeptide in the presence of said stress-inducing agent or condition is manifest as
(i) a morphological change,
(ii) cell growth inhibition, or
(iii) lysis
of the cells expressing the polypeptide compared to morphology, growth or survival of said cells treated with the stress inducing agent or condition in the absence of the candidate anti-fungal compound.

58. The screening method according to claim 57, wherein the cell wall stress inducing agent or condition is selected from the group consisting of: calcofluor white, caspofungin, tunicamycin, sodium dodecyl sulfate, and elevated temperature.

59. The screening method according to claim 57, wherein the effect of the candidate antifungal compound and stress-inducing agent or condition is assessed by measuring cell growth.

60. The screening method according to claim 59, wherein cell growth in the presence of the candidate anti-fungal compound is reduced by at least 10% compared to the growth of said cells treated with said stress inducing agent or condition in the absence of the candidate anti-fungal compound.

61. A method for producing a polypeptide having UDP-galactopyranose mutase enzymatic activity, wherein the amino acid sequence of the polypeptide is at least 48% identical with SEQ ID NO:1, comprising culturing the host cell according to claim 50 under suitable culture conditions.
